(19) United States
(12) Patent Application Publication
(10) Pub. No.: US 2026/0013890 A1
(43) Pub. Date: Jan. 15, 2026

(54) MEDICAL INSTRUMENT

(71) Applicant: KARL STORZ SE & Co. KG, Tuttlingen (DE)

(72) Inventor: Martin HOFFMANN, Berlin (DE)

(73) Assignee: KARL STORZ SE & Co. KG, Tuttlingen (DE)

(21) Appl. No.: 19/265,128

(22) Filed: Jul. 10, 2025

(30) Foreign Application Priority Data
Jul. 10, 2024 (DE) 10 2024 119 580.0

(51) Int. Cl. A61B 17/29; A61B 17/00

(52) U.S. Cl. CPC A61B 17/29; A61B 2017/00477; A61B 2017/2902; A61B 2017/2927

(57) ABSTRACT

The disclosure relates to a medical instrument comprising: two drive elements which can be moved at least along their longitudinal direction, and an end effector which has at least two cooperating end effector elements, wherein the end effector elements are each coupled to one of the drive elements in a movement-transmitting manner and are pivotable about a pivot axis independently of one another.

### CROSS-REFERENCE TO RELATED APPLICATIONS

This application claims priority of German Patent Application No. DE 10 2024 119 580.0 filed on Jul. 10, 2024, the contents of which is incorporated herein.

### TECHNICAL FIELD

The present disclosure relates to a medical instrument.

### BACKGROUND

Due to the reliable and precise control thereof, medical robot systems and/or at least partially electrically operated medical instruments are increasingly replacing conventional, manually operable medical instruments. In order to be able to provide the required flexibility and/or articulation of medical instruments, currently known medical instruments have an extremely complex and/or cost-intensive design. Furthermore, conventional medical instruments used in robot systems have thermally unstable components, for example in the form of cable arrangements, so that reuse of such medical instruments, in particular after one or more sterilization processes, is only possible to a limited extent. Since regular new purchases are therefore necessary, not only the ecological but also the economic sustainability of such medical instruments is significantly impacted.

The object of the disclosure is, but is not limited to, advantageously developing a medical instrument, in particular for use in a medical robot system, primarily with respect to increasing freedom of movement and reliability. Furthermore, it is an object of the present disclosure, inter alia, to ensure reusability of such a medical instrument and therefore reduce operating costs.

This object is achieved according to the disclosure by the features of the independent claims. Developments of the disclosure can be found in the dependent claims.

### SUMMARY

The disclosure relates to a medical instrument comprising:
two drive elements which can be moved at least along their longitudinal direction, and
an end effector which has at least two cooperating end effector elements, the end effector elements each being coupled to one of the drive elements in a movement-transmitting manner and being pivotable about a pivot axis independently of one another.

Such a design makes it possible to provide an advantageously developed medical instrument. In particular, such a medical instrument can have a high degree of freedom of movement and reliability. In addition, costs for medical applications can be reduced because a medical instrument of this kind can be reused.

A “medical instrument” is to be understood in particular to be a medical tool which is preferably designed to grip, manipulate, hold, cut and/or otherwise interact with an object to be handled.

The term “designed” is to be understood to mean specifically programmed, provided, configured, formed, and/or equipped. The fact that an object is designed for a specific function is to be further understood to mean that the object fulfills and/or carries out this specific function in at least one application and/or operating state.

The medical instrument can be intended for use in surgical procedures and/or invasive operations. The medical instrument can be part of a medical robot system and/or can at least be functionally coupled to such a system. In some embodiments, the medical instrument can be designed as a hand-held medical instrument.

An object to be handled can refer to any organic and/or inorganic structure. In particular, this means anatomical structures of a patient, such as organs and/or tissues, and/or consumables such as threads, staples, films, swabs, tubes, screws and/or nails.

A drive element is preferably intended to be a component and/or device which is designed to generate, transmit and/or control movements.

An “end effector” is to be understood in particular to be a component and/or device of the medical instrument which is preferably arranged at the distal end of the medical instrument, or in other words, close to the patient during use of the medical instrument. The end effector is designed to establish physical contact with and/or interact with one and/or more objects to be handled. The end effector can be designed differently depending on the range of requirements and/or task.

“At least two cooperating effector elements” are to be understood to be at least two subcomponents of the end effector which interact with, cooperate with and/or mutually influence one another in order to carry out and/or fulfill a specific task. The two effector elements can be designed to be complementary. The effector elements can form a form-fitting engagement.

The fact that the end effector elements can be pivoted about at least one pivot axis independently of one another can mean that the two end effector elements can be pivoted separately and/or without being directly influenced by a movement of the other effector element. The pivot axis can extend perpendicularly to the longitudinal direction of the drive elements. The pivot axis can be a reference axis which can relate to a single degree of freedom which can allow a pivoting movement of the end effector elements about said pivot axis. The pivoting movement of the end effector elements can occur in predefined angular steps, but is preferably almost continuous and particularly preferably continuous. Each of the end effector elements can be pivotable by at least 45°, preferably at least 90° and particularly preferably at least 160°, about the pivot axis. The end effector elements can be designed as jaw parts.

According to a development, the medical instrument can comprise at least two output elements, each of which is connected to one of the end effector elements for conjoint rotation. The output elements can each be coupled to one of the drive elements in a movement-transmitting and/or movement-transmittable manner. The output elements can be designed to absorb forces and/or movements of the drive elements. Therefore, a reliable and/or precise movement transmission between the drive elements and the output elements and/or the end effector elements can be ensured.

In some embodiments, at least one of the output elements can be designed as a pinion. At least one of the drive elements can be designed as a rack which cooperates with one of the output elements and/or with the pinion. In some embodiments, the output elements and the end effector elements can be formed in one-piece and/or monolithically. The pinions and/or racks can preferably be made of thermally stable materials, such as metals and/or high performance polymers, such as polyether ketones. In some embodiments, the drive and/or output elements can also comprise other mechanical components and/or drive mechanisms that appear advantageous to a person skilled in the art, such as a worm gear and/or a Maltese cross gear. By means of such a design, movements can be transmitted reliably and/or precisely by rolling movements. Such a design can be robust against wear and/or high temperatures, significantly increasing the service life of the medical instrument as a result, despite sterilization processes at high temperature ranges and/or frequent use.

According to a development, the medical instrument can comprise a shaft which has a distal shaft portion in which the drive elements are arranged at least in portions. The shaft can be an elongate and/or cylindrical component. Mechanical components and/or cables can be guided securely and/or in an organized manner through the shaft. The shaft can contribute to the stability of the medical instrument and/or protect kinematic structures against external influences.

In some embodiments, the medical instrument can comprise at least two actuator elements. Each of the actuator elements can be coupled to one of the drive elements in a movement-transmitting and/or movement-transmittable manner. The actuator elements can be arranged, at least in portions, in a proximal shaft portion of the shaft. A proximal shaft portion can refer to a portion of the shaft of the medical instrument that faces away from the patient during operation. Each of the actuator elements can be formed as a pull and/or push rod which is designed to transmit pull and/or push forces to a relevant drive element. In other words, it would be conceivable that each actuator element can be moved in two opposite directions along a longitudinal axis of the shaft. Such pull and/or push rods provide increased stability, efficient force transmission and/or better load distribution, which can contribute to improved performance, reliability and/or service life of the medical instrument.

According to a development, the medical instrument can comprise at least two coupling elements, each of which couples one of the drive elements to one of the actuator elements in an articulated, captive, movement-transmitting and/or movement-transmittable manner. The coupling elements can be arranged in parallel, at least in portions. In this way, the end effector and/or the end effector elements can be efficiently and/or reliably controlled over a distance and/or coupled to one another.

In addition, the shaft of the medical instrument can comprise a hinge joint, by means of which the distal shaft portion can be bent about a joint axis relative to the proximal shaft portion. The coupling elements can be arranged in a region of the hinge joint. This design gives the medical instrument an additional degree of freedom. The additional degree of freedom allows the distal shaft portion to be able to pivot about the joint axis relative to the proximal shaft portion. The distal shaft portion can be pivotable and/or bendable about the joint axis by at least 45°, preferably at least 90° and particularly preferably at least 160°. This joint axis can extend perpendicularly to the pivot axis about which the end effector elements can be pivoted. The additional degree of freedom can improve the freedom of movement of the medical instrument and/or the precision. This can also contribute to better maneuverability, extended reach, and/or more efficient performance of surgical procedures, in particular in difficult-to-access regions such as patient cavities. The alignment and/or control of the hinge joint and/or of the distal shaft portion can occur independently of the control and/or alignment of the end effector and/or of the end effector elements.

In some embodiments, a distance between the pivot axis and the joint axis can be constant during use of the medical instrument, reducing and/or preventing inadvertent and/or parasitic movements of the shaft portions and/or the end effector elements as a result.

The devices according to the disclosure are not to be limited to the application and embodiment described above. In particular, they can have a number of individual elements, components, and units which differ from a number stated herein, in order to fulfill a function described herein. In addition, for the ranges of values specified in this disclosure, values within the stated limits shall also be deemed to be disclosed and to be usable in any manner.

The present disclosure is described below by way of example with reference to the accompanying drawings. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the drawings and in the description. The description of this example can be transferred correspondingly to the other examples of the object. If objects are named using numerical words, such as first, second, third object, etc., these are used to name and/or assign objects. Accordingly, for example, a first object and a third object may be included, but not a second object. However, a number and/or sequence of objects could additionally be derived using numerical words.

### BRIEF DESCRIPTION OF DRAWINGS

FIG. 1 is a perspective view of a medical instrument which has a kinematic structure,

FIG. 2 is a detailed view of the kinematic structure of the medical instrument in a first position,

FIG. 3 is a detailed view of the kinematic structure of the medical instrument in the first position,

FIG. 4 is a detailed view of one half of the kinematic structure of the medical instrument,

FIG. 5 is a detailed view of the kinematic structure of the medical instrument in a second position,

FIG. 6 is a side view of the kinematic structure of the medical instrument according to FIG. 5,

FIG. 7 is a side view of the kinematic structure of the medical instrument in a third position,

FIG. 8 is a detailed view of an end effector in a first position,

FIG. 9 is a detailed view of an end effector in a second position, and

FIG. 10 is a side view of a medical instrument according to an additional embodiment.

### DETAILED DESCRIPTION OF PREFERRED EMBODIMENTS

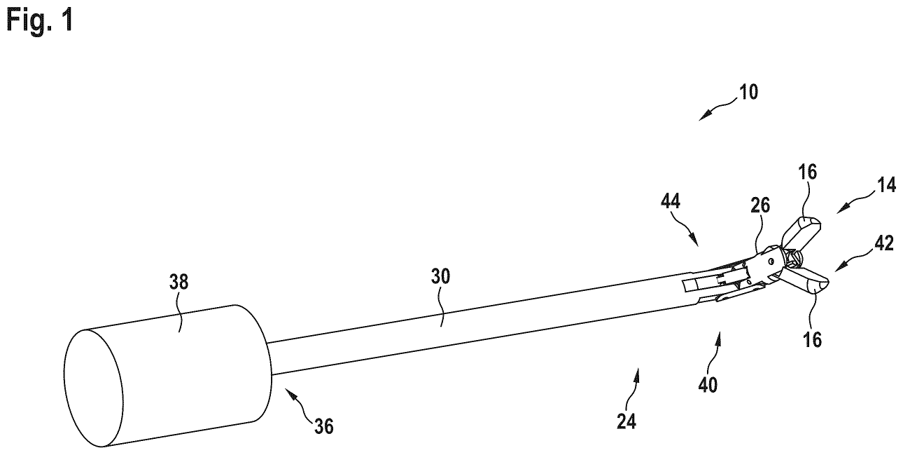

In FIG. 1, a medical instrument 10 is shown having a shaft 24 which has a proximal shaft portion 30 and a distal shaft portion 26. An interface 38 of the medical instrument 10 is arranged at a proximal end 36 of the proximal shaft portion 30. The interface 38 can be designed to be coupled in a functional and/or controllable manner to a robot device (not shown herein) and/or to a handle. The distal shaft portion 26 of the medical instrument 10, which has an end effector 14, is arranged at a distal end 40 of the proximal shaft portion 30. The end effector 14 comprises a gripping arrangement 42 which has two end effector elements 16.

At this point, it should be noted that the gripping arrangement 42 is to be understood only by way of example and, depending on the field of application, other end effectors and/or arrangements that appear advantageous to a person skilled in the art can also be provided. A length of the proximal shaft portion 30 can correspond in particular to at least five times, preferably at least ten times and particularly preferably at least fifteen times the length of the distal shaft portion 26.

The end effector 14, the end effector elements 16 and/or the distal shaft portion 26 can be controlled and/or moved by means of a kinematic structure 44 of the medical instrument 10.

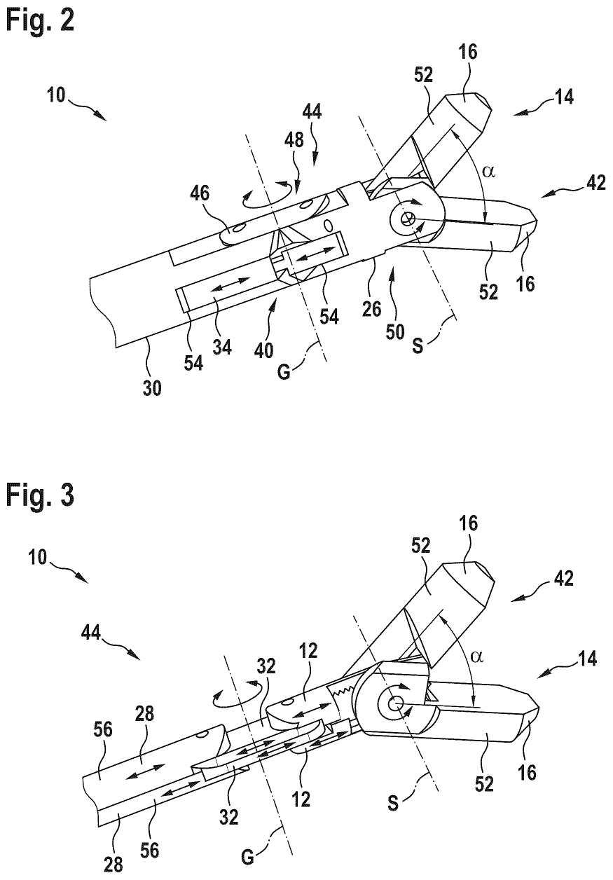

In FIG. 2, a detailed view of the kinematic structure 44 in a first position is shown. The distal shaft end 40 of the proximal shaft portion 30 can be seen, which is captively and movably coupled to a proximal shaft end 48 of the distal shaft portion 26 by means of two tab elements 46. Only one of the tab elements 46 is visible, the other tab element 46 is hidden in this view. The tab elements 46 space the distal shaft portion 26 apart from the proximal shaft portion 30 and ensure a constant distance between the distal shaft end 40 of the proximal shaft portion 30 and the proximal end 48 of the distal shaft portion 36.

Furthermore, the medical instrument 10 and/or the kinematic structure 44 comprises a hinge joint 34, by means of which the distal shaft portion 26 and a joint axis G can be bent relative to the proximal shaft portion 30. The hinge joint 34 is guided in and/or along a guiding groove 54 formed on the proximal shaft portion 30 and the distal shaft portion 26. A pivot axis S, about which each of the end effector elements 16 can be pivoted independently of one another, and the joint axis G are indicated by dashed lines. Degrees of freedom of the end effector elements 16 and/or of the distal shaft portion 26 achieved by the joint axis G and/or the pivot axis S are indicated as arrows.

The end effector 14 is arranged at a distal shaft end 50 of the distal shaft portion 26 and has two end effector elements 16. In the embodiment shown herein, the two end effector elements 16 are each configured as a jaw part 50. The two jaw parts 52 are designed to be complementary so that they can cooperate with the other jaw part 52. The two jaw parts 52 can be pivoted about the pivot axis S independently of one another. In the position shown herein, the two end effector elements 16 are pivoted relative to one another about the pivot axis S at a first angle α. The pivot axis S and the joint axis G extend perpendicularly to one another.

The detailed view of the kinematic structure 44 shown in FIG. 3 differs from the view shown in FIG. 2 in that in FIG. 3, the proximal shaft portion 30, the distal shaft portion 26 and the hinge joint 34 are hidden. Without the shaft portions 30, 26, additional elements of the kinematic structure 44 can be seen. Two drive elements 12 of the kinematic structure 44 can be seen, which are coupled in a movement-transmitting and/or movement-transmittable manner to output elements 18 arranged on the end effector elements. The output elements 18 and/or the drive elements 12 are arranged in a region of the distal shaft portion 26.

In the embodiment shown here, the drive elements 12 are designed as racks 22. Two actuator elements 28 of the kinematic structure 44 can also be seen, which are designed herein as pull and/or push rods 56. The two actuator elements 28 are arranged in a region of the proximal shaft portion 26 and extend in parallel with one another. Each of the two actuator elements 28 can move in a linear movement independently of the other actuator element 28. Degrees of freedom of the actuator elements 28 are correspondingly indicated by arrows.

Each of the two actuator elements 28 is coupled in an articulated manner to one of the drive elements 12, in each case via a coupling element 32, in a movement-transmitting and/or movement-transmittable manner.

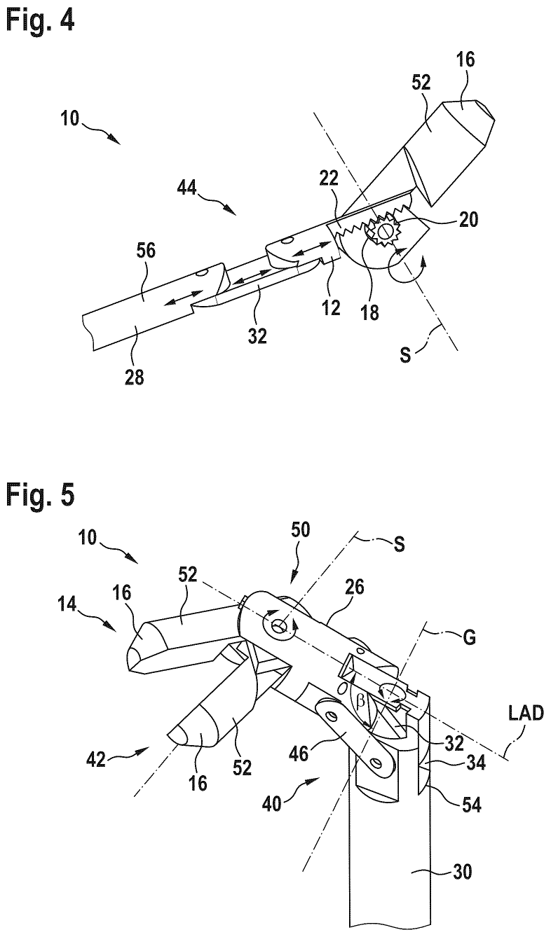

FIG. 4 is a detailed view of one half of the kinematic structure 44 of the medical instrument 10. In this figure, in comparison to FIG. 3, only an actuator element 28, a coupling element 32, a drive element 12 and an end effector element 16 is shown. Furthermore, the output element 18 can be seen in FIG. 4, which is designed herein in the form of a pinion 20 and is coupled to the rack 22 in a movement-transmitting and/or movement-transmittable manner. The degrees of freedom of the individual components are to be schematically indicated by arrows. A linear movement of the actuator element 28 moves the coupling element 32 and the drive element 12 in the same direction, as a result of which the linear movement of the drive element 12 is ultimately transformed into a rotational movement of the output element 18 at the point at which the drive element 18 is coupled to the output element 18.

FIG. 5 is a detailed view of the kinematic structure 44 of the medical instrument 10 in a second position. The difference from the position and/or state of the kinematic structure 44 shown in FIGS. 1 to 4 is the orientation of the proximal shaft portion 30 relative to the distal shaft portion 26. In the second state shown, the hinge joint 34 has been pushed in the direction of the distal shaft portion 26 so that the distal shaft portion 26 is bent at a second angle β about the joint axis G relative to the proximal shaft portion 30. The guiding grooves 54, in which the hinge joint 34 is guided, are delimited both on the side of the proximal shaft portion 30 and on the side of the distal shaft portion 26. This limitation can act as a type of stop element which allows and/or delimits movement of the hinge joint 34 in the one and/or the other direction.

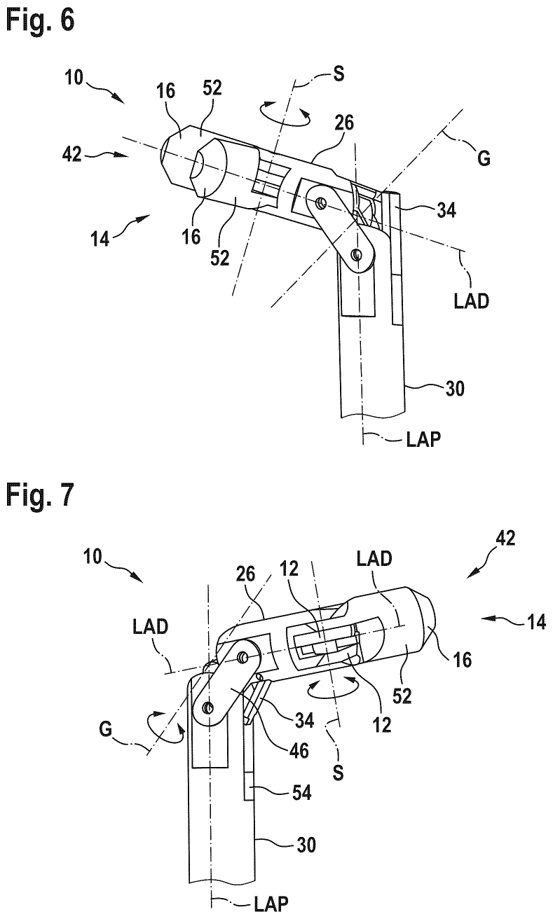

FIGS. 6 and 7 show a pivoting of the distal shaft portion 26 about the joint axis G. The position and/or state in which the kinematic structure 44 is located in FIG. 6 corresponds to the position shown in FIG. 5. By moving the hinge joint 34 in the direction of the distal shaft portion 26, the distal shaft portion 26 and the hinge joint 34 and/or the longitudinal axis LAD of the distal shaft portion are bent by an angular amount relative to the longitudinal axis LAP of the proximal shaft portion. The end effector elements are pivoted by an angular amount relative to the longitudinal axis of the distal shaft portion 26 by positioning of the actuator elements 28 and/or the drive elements 12.

In FIG. 7, the hinge joint 34 has been moved in the opposite direction, i.e. in the direction of the proximal shaft end 36 of the proximal shaft portion 30, so that the distal shaft portion has been pivoted in the opposite direction by approximately 160°.

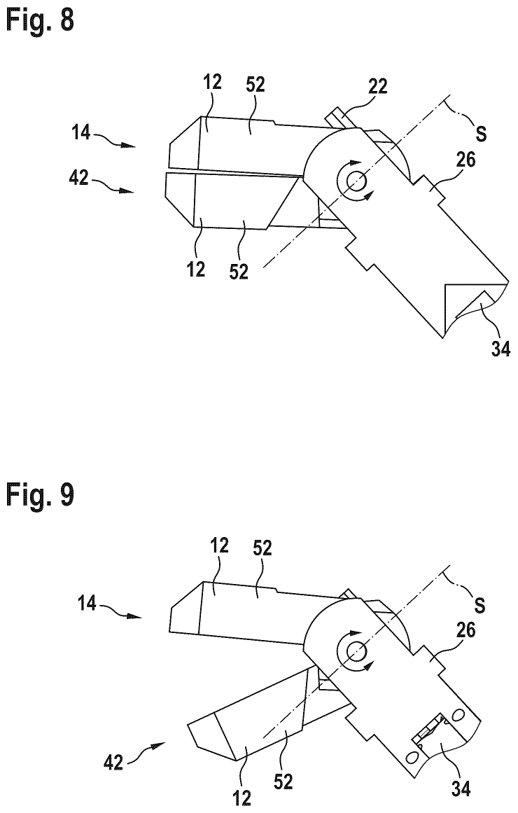

FIGS. 8 and 9 are a detailed view of an end effector and/or the end effector elements in different positions and/or states. According to FIG. 8, the two end effector elements 16 designed as jaw parts 52 are aligned in a closed and/or gripping position. The end effector elements 16 are designed to be substantially form-fitting, as a result of which a high clamping force is ensured. By controlling and/or moving the actuator elements 28 and the movements of the drive elements 12 resulting therefrom, the end effector elements 16 can be pivoted independently of one another and/or relative to one another into an open and/or release position shown in FIG. 9.

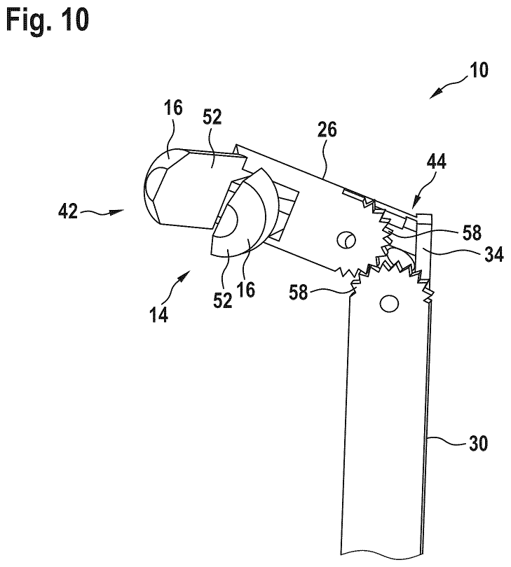

FIG. 10 shows an additional embodiment of a medical instrument. According to this embodiment, the shaft ends 40, 48 of the proximal and the distal shaft portion 26, 30 are designed as pinion profiles 58 rolling on one another. The design as pinion profiles 58 rolling on one another is a particularly simple design solution which, due to the form-fitting and frictional engagement occurring between the teeth on both sides, makes it possible to move the distal shaft portion 26 securely and/or reliably into a variety of angled positions and/or to hold said shaft portion in desired positions. The tab elements 46 are hidden in FIG. 10 for the sake of clarity.

1. Medical instrument comprising:
two drive elements which can be moved at least along their longitudinal direction, and
an end effector which has at least two cooperating end effector elements,
wherein the end effector elements are each coupled to one of the drive elements in a movement-transmitting manner and are pivotable about a pivot axis independently of one another.

2. Medical instrument according to claim 1, further including at least two output elements which are each connected to one of the end effector elements for conjoint rotation, wherein the output elements are each coupled to one of the drive elements in a movement-transmitting manner.

3. Medical instrument according to claim 2, wherein at least one of the output elements is designed as a pinion and at least one of the drive elements is designed as a cooperating rack.

4. Medical instrument according to claim 1, further including a shaft which has a distal shaft portion in which the drive elements are arranged at least in portions.

5. Medical instrument according to claim 4, further including at least two actuator elements, wherein each of the actuator elements is coupled to one of the drive elements in a movement-transmitting manner, and wherein the actuator elements are arranged, at least in portions, in a proximal shaft portion of the shaft.

6. Medical instrument according to claim 5, further including at least two coupling elements which in each case couple one of the drive elements to one of the actuator elements in an articulated and movement-transmitting manner.

7. Medical instrument according to claim 6, wherein the shaft includes a hinge joint by means of which the distal shaft portion can be bent about a joint axis relative to the proximal shaft portion, and wherein the coupling elements are arranged in a region of the hinge joint.

8. Medical instrument according to claim 7, wherein a distance between the one pivot axis and the joint axis is constant.
